(19) United States
(12) Patent Application Publication
(10) Pub. No.: US 2007/0004691 A1
(43) Pub. Date: Jan. 4, 2007

(54) Antitumoral combinations containing et-743 and a cruciferous and a cruciferous indole compound

(76) Inventors: Sarah Donald, Leicestershire (GB); Andreas Gescher, Leicestershire (GB); Jose Maria Jimeno Donaque, Madrid (ES)

Correspondence Address: MORGAN & FINNEGAN, L.L.P., 3 WORLD FINANCIAL CENTER, NEW YORK, NY, 10281-2101, US

(21) Appl. No.: 10/575,132

(22) PCT Filed: Oct. 14, 2004
(86) PCT No.: PCT/GB04/04358

§ 371 Date: Jul. 7, 2006

(30) Foreign Application Priority Data
Oct. 15, 2003 (GB) 0324201.3

(51) Int. Cl. A61K 31/573; A61K 31/405

(52) U.S. Cl. 514/171; 514/419; 514/514

(57) ABSTRACT

Administration of a cruciferous indole compound can reduce undesirable toxic side effects inherent in the anti-tumor therapy with ET-743 before during or after administration of the ET-743.

### FIELD OF THE INVENTION

This invention relates to a combination drug treatment and a method for treating patients afflicted with cancer, having fewer and less severe unwanted toxic adverse effects.

### BACKGROUND OF THE INVENTION

Ecteinascidin 743 (ET-743) is a tetrahydroisoquinoline alkaloid isolated from the marine tunicate Ecteinascidia turbinata with the following structure:

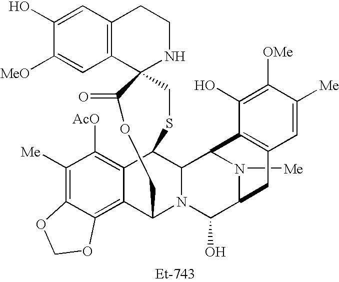

A recent review of ET-743, its chemistry, mechanism of action and preclinical and clinical development can be found in Kesteren, Ch. Van et al., 2003, Anti-Cancer Drugs, 14 (7), pages 487-502: “Yondelis (trabectedin, ET-743): the development of an anticancer agent of marine origin”, and references therein.

ET-743 possesses potent antineoplastic activity against a variety of human tumour xenografts grown in athymic mice, including melanoma and ovarian and breast carcinoma.

In clinical phase I studies of ET-743, promising responses were observed in patients with sarcoma and breast and ovarian carcinoma. Therefore this new drug is currently under intense investigation in several phase II clinical trials in cancer patients with a variety of neoplastic diseases.

ET-743 has myelotoxic and hepatotoxic side effects. Patients who received ET-743 by prolonged infusion over 24-72 hr experienced myelosuppression and, frequently, acute, albeit reversible, elevation of transaminases and subclinical cholangitis characterized by increases in alkaline phosphatase (ALP) and/or bilirubin, see for example Ryan D. P. et al., 2001 Clin Cancer Res 7, 231: “Phase I and pharmacokinetic study of ecteinascidin 743 administered as a 72-hour continuous intravenous infusion in patients with solid malignancies”; Puchalski T. A. et al., 2002, Cancer Chemother Pharmacol 50: 309: “Pharmacokinetics of ecteinascidin 743 administered as a 24-h continuous intravenous infusion to adult patients with soft tissue sarcomas: associations with clinical characteristics, pathophysiological variables and toxicity”.

Preclinical acute toxicity studies conducted in mice, rats, dogs and monkeys consistently demonstrated liver toxicity as an important side effect of ET-743, as evidenced by an increase in plasma levels of liver-specific enzymes and pathological manifestations of cholangitis. Recently the nature and extent of the hepatobiliary changes exerted by ET-743 in the female rat, the species which is most susceptible towards the hepatotoxic potential of ET-743, has been characterized by histopathology, electron microscopy, immunohistochemistry, plasma biochemistry and DNA microarray analysis, see Donald S. et al., 2002, Cancer Research, 62: 4256 “Hepatobiliary damage and changes in hepatic gene expression caused by the antitumor drug ecteinascidin 743 (ET-743) in the female rat”.

Furthermore, pretreatment with high-dose dexamethasone has been shown to abrogate ET-743-mediated hepatotoxicity in this animal model without impeding its antitumor activity, see Donald S. et al., 2003, Cancer Research, 63: 5903-5908: “Complete protection by high-dose dexamethasone against the hepatotoxicity of the novel antitumor drug ecteinascidin-743 (ET-743) in the rat” and also see WO 02 36135. Protection by dexamethasone pretreatment was accompanied by a dramatic reduction in hepatic levels of ET-743, tentatively implicating elevated hepatic clearance of ET-743, perhaps via induced metabolic enzymes, as the mechanism by which dexamethasone exerts its beneficial effect, i.e. via an increase in the rate of metabolic detoxification of ET-743.

However, the potential use of high-dose dexamethasone as a hepatoprotectant in humans has the problem that it might be confounded by adverse effects, including diabetes mellitus, hypertension, arrhythmias, hypokalemia, psychosis and susceptibility to infection.

Therefore there is still a need to provide further therapies that allow an effective treatment of mammals, in particular humans, with ET-743 while reducing or eliminating its toxic side effects, in particular the liver toxicity and minimizing further adverse effects.

### SUMMARY OF THE INVENTION

We have now found that cruciferous indole compounds, and in particular indole-3-carbinol are suitable hepatoprotectants for ET-743 without the above mentioned adverse effects. Indeed, as the examples show, a combination of indole-3-carbinol with ET-743 almost completely abolished manifestations of hepatotoxicity, while not interfering with the antitumor efficacy of ET-743.

In one aspect, the present invention is directed to a composition for the treatment of cancer, comprising ET-743 and a cruciferous indole compound, which is effective in reducing the hepatotoxic side effects of ET-743. Preferably the cruciferous indole compound is indole-3-carbinol or derivatives thereof, most preferably indole-3-carbinol also referred to herein as I3C.

In another aspect, the present invention is directed to the use of ET-743 in the preparation of a medicament for an effective treatment of a tumour by combination therapy employing ET-743 with a cruciferous indole compound. Preferably the cruciferous indole compound is Indole-3-carbinol or derivatives thereof, most preferably Indole-3-carbinol. The treatment is effective in reducing the hepatotoxic side effects of ET-743.

The invention also provides a method of treating a mammal affected by a tumor comprising administering an effective therapeutic amount of a cruciferous indole compound, preferably indole-3-carbinol, in combination with ET-743. Preferably the mammal is a human. The method is effective in reducing the hepatotoxic side effects of ET-743.

In a preferred embodiment the indole cruciferous compound is given in combination with another hepatoprotector, preferably dexamethasone.

In a preferred aspect the cruciferous indole compound is administered prior to ET-743, preferably at least 3 days, more preferably 5 days, prior to the treatment with ET-743. In a most preferred aspect the cruciferous indole compound, in particular I3C is given at least 6 days prior to the treatment with ET-743.

In a preferred method the indole-3-carbinol is administered in a dosage in the range of about 0.02-5 g/m² body surface area, more preferably about 0.5-3 g/m² body surface area. Particularly preferred is a dosage of about 2,6 g/m² body surface area. These dosages are particularly suitable for indole-3-carbinol as the cruciferous indole compound. Alternatively the I3C is preferably administered at a dose of 200-500 mg per day. In additional embodiments, I3C is administered at doses of 200-300 mg per day, 300-400 mg per day and 400-500 mg per day. If given in combination with another hepatoprotectant such as dexamethasone these amounts can be reduced.

Preferably the indole-3-carbinol is administered orally.

### DETAILED DESCRIPTION OF THE INVENTION

We have found that administration of cruciferous indole compounds, in particular indole-3-carbinol, strongly protects the liver from ET-743 induced damage, without displaying the detrimental side effects of high-dose dexamethasone.

This is the more surprising in view that, besides dexamethasone, other modulators of drug metabolism have not shown protective capacities. Indeed, the hypothesis was also tested that β-naphthoflavone, phenobarbitone and N-acetylcysteine, three well-characterized modulators of drug metabolism, protect the liver against ET-743. β-Naphthoflavone, phenobarbitone and dexamethasone induce cytochrome P450 enzyme families CYP1A1/2, 2B and 3A, respectively, and thus can increase the rate of oxidative metabolic disposition of suitable drug substrates. The hepatoprotective capacities of β-naphthoflavone, phenobarbitone and N-acetylcysteine were compared with that of dexamethasone. We found that amelioration of ET-743 mediated hepatotoxicity by β-naphthoflavone persisted only for a short term, and that by phenobarbitone was weak, as reflected by significant suppression of elevation of only one biochemical indicator, bilirubin, but not of the others. The other agent, N-acetylcysteine, failed to protect rat livers against ET-743 altogether.

These findings complement our recent detailed characterization of dexamethasone as a potent antidote against ET-743-mediated hepatotoxicity in the female rat. The protection afforded by dexamethasone was accompanied by dramatically decreased hepatic levels of ET-743 and by up-regulated CYP3A enzyme levels, suggesting that protection by dexamethasone is the corollary of the increased clearance of the drug from the liver, possibly mediated by metabolism involving CYP3A.

Indole-3-carbinol (I3C) (1H-indole-3-methanol; 3-(hydroxy methyl)indole) has the following formula:

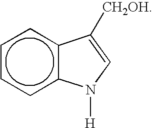

It is a microconstituent of cruciferous vegetables such as broccoli and Brussels sprouts. These vegetables contain μg/g levels of glucobrassicin, an indolylmethyl glucosinolate. When the plant cells are damaged as by cutting or chewing, a thioglucosidase-mediated autolytic process takes place generating I3C, glucose and thiocyanate ion. At acid pH, I3C forms a wide variety of condensation products ranging from linear and cyclic dimers, trimers, and tetramers to extended heterocyclic compounds such as indolocarbazoles.

Indole-3-carbinol has chemopreventive properties against chemically induced tumors in rodents at a variety of sites. Clinical pilot studies of I3C have been conducted in patients with recurrent respiratory papillomatosis, cervical intraepithelial neoplasia, and there has been a dose-finding study to prepare its evaluation as a breast cancer preventive agent. I3C is a non-toxic diet constituent and a potent inducer of cytochrome P450 enzymes.

Other hepatoprotective cruciferous indole compounds or derivatives thereof can also be used in the invention, instead of indole-3-carbinol: compounds such as 5-methyl-indole-3-carbinol, 5-ethyl-indole-3-carbinol, 5-propyl-indole-3-carbinol, 5-butyl-indole-3-carbinol, 5-pentyl-indole-3-carbinol, 5-methoxy-indole-3-carbinol, 5-ethoxy-indole-3-carbinol, 5-propyloxy-indole-3-carbinol, 5-butyloxy-indole-3-carbinol, 5-amyloxy-indole-3-carbinol, N-methyl-indole-3-carbinol, N-ethyl-indole-3-carbinol, N-propyl-indole-3-carbinol, N-butyl-indole-3-carbinol, N-pentyl-indole-3-carbinol, 2-methyl-indole-3-carbinol, 2-ethyl-indole-3-carbinol, 2-propyl-indole-3-carbinol, 2-butyl-indole-3-carbinol and 2-pentyl-indole-3-carbinol. Further examples of the compounds that can be used are given in U.S. Pat. No. 6,369,095 incorporated herein by reference in its entirety.

The cruciferous indole compounds and the pharmaceutically acceptable salts thereof can be administered to a host in any effective way such as via oral, parenteral or topical routes. Generally, the compounds are administered in dosages ranging from about 2 mg up to about 7 g per day, although variations will necessarily occur depending on the disease target, the host, and the route of administration. Preferred dosages are administered orally in the range of about 0.02-5 g/m² body surface area, more preferably about 0.5-3 g/m² body surface area. Particularly preferred is a dosage of about 2.6 g/m² body surface area. These dosages are particularly suitable for indole-3-carbinol as the cruciferous indole compound. The I3C is also preferably administered at a dose of 200-500 mg per day. In alternative embodiments, I3C is administered at doses of 200-300 mg per day, 300-400 mg per day and 400-500 mg per day. If given in combination with another hepatoprotectant such as dexamethasone the amount given can be reduced.

The dose of cruciferous indole compound is given periodically, preferably daily. Most preferably it is given orally as a diet supplement. In a preferred aspect of the invention the cruciferous indole compound is given prior to the treatment with ET-743, preferably at least 3 days, more preferably 5 days, prior to the treatment with ET-743. In a most preferred aspect the cruciferous indole compound, in particular 13C is given at least 6 days prior to the treatment with ET-743.

The indole cruciferous compounds can be combined with a pharmaceutically acceptable excipient such as sterile saline or other medium, gelatine, an oil, etc. to form pharmaceutically acceptable compositions. The compositions and/or compounds may be administered alone or in combination with any convenient carrier, diluent, etc. and such administration may be provided in single or multiple dosages. Useful carriers include solid, semi-solid or liquid media including water and non-toxic organic solvents. The indole cruciferous compounds can also be administered in the form of a pro-drug, which can be metabolically converted to the subject compound by the recipient host. A wide variety of pro-drug formulations are known in the art. The compositions comprising indole cruciferous compounds, in particular indole-3-carbinol may be provided in any convenient form including tablets, capsules, lozenges, troches, hard candies, powders, sprays, creams, suppositories, etc. As such the compositions, in pharmaceutically acceptable dosage units or in bulk, may be incorporated into a wide variety of containers. For example, dosage units may be included in a variety of containers including capsules, pills, etc.

The indole cruciferous compounds may be advantageously combined and/or used in combination with other hepatoprotective agents, different from the subject compounds. In many instances, administration in conjunction of the hepatoprotectants enhances the efficacy of such agents. For example, the compounds, in particular I3C, may be advantageously used in conjunction with dexamethasone in order to reduce the dose needed of each compound, to obtain adequate hepatoprotection against the side effects of ET-743 while at the same time reducing the adverse effects of high doses of any of them.

The term “ET-743” is intented here to cover any pharmaceutically acceptable salt, ester, solvate, hydrate or any other compound which, upon administration to the recipient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art.

ET-743 is supplied and stored as a sterile lyophilied product, consisting of ET 743 and excipient in a formulation adequate for therapeutic use, in particular a formulation containing mannitol and a phosphate salt buffered to an adequate pH.

The administration of ET-743 is performed in cycles, in the application method of the invention, an intravenous infusion of ET-743 is given to the patients every week, allowing for a resting phase in each cycle in which the patients recover. Preferably the resting phase is one week within each cycle. The preferred duration of each cycle is of 2 to 4 weeks; multiple cycles can be given as needed. Dose delays and/or dose reductions and schedule adjustments are performed as needed depending on individual patient tolerance of treatments. The infusion time is between 1 and 24 hours, preferably between 1 and 3 hours. Especially preferred is a time of about 3 hours. The preferred schedules of administration of ET-743 are 24 hour every 3 weeks, 3 hour every 3 weeks and 1-3 hours iv infusion for 3 consecutive weeks every 4 weeks.

The recommended dose for the 24 hour q3W schedule is 1500 μg/m² and the recommended dose for the 3 hour q3W schedule is at present between 1300 and 1650 μg/m². The recommended dose for the 1-3 hour weekly schedule is between 300 and 600 μg/m²/weekly, preferably about 580 μg/m²/weekly.

Further guidance on the dosage, schedules and administration of ET-743 alone or in combination is given in WO 00 69441, WO 02 36135, WO 03 039571 and PCT/GB2004/002319 which are each incorporated by reference herein in their entirety.

The combination of ET-743 and a cruciferous indole compound allows for an effective cancer therapy in humans, while avoiding liver toxicities. For example ET-743 together with the indole cruciferous compound, preferably I3C, is used for the treatment of sarcoma, osteosarcoma, ovarian cancer, breast cancer, melanoma, colorectal cancer, mesothelioma, renal cancer, endometrial cancer and lung cancer, as a first line therapy or for patients relapsing or refractory after previous chemotherapeutic treatments.

Depending on the type of tumour and the developmental stage of the disease, the treatments of the invention are useful in preventing the risk of developing tumours, in promoting tumour regression, in stopping tumour growth and/or in preventing metastasis.

The following examples will further illustrate the invention. They should not be interpreted as a limitation of the scope of the invention.

### EXAMPLES

### Example 1

### Study of Hepatotoxicity

I3C at 0.1 or 0.5% or di-indolyl methane (DIM) at 0.2% were added to the diet of female Wistar rats (230-260 g) seven days prior to treatment with a hepatotoxic dose of ET-743 (40 μg/kg, iv. via the lateral tail vein). This amount constitutes doses equivalent to approximately 80 and 400 mg/kg I3C per day. Control animals received the unaltered diet and/or water as the vehicle for ET-743. The dose of DIM was based on the following consideration: DIM is the product of the condensation of two molecules of I3C, therefore assuming all of the I3C in the 0.5% dose group dimerises to form DIM, the maximal amount of DIM which could theoretically be generated is half of the amount of I3C in the diet. Each treatment group comprised 4 animals. Hepatic changes were studied by assessment of alterations in plasma levels of bilirubin and liver enzymes ALP and aspartate aminotransferase (AST) and by conventional histopathological investigation of liver tissue. Day three is the time point at which liver damage was found to be maximal in rats which received ET-743 alone. ET-743 alone elicited hepatic alterations identical with those reported earlier, as borne out by dramatically raised levels of bilirubin, elevated activities of ALP and AST and liver pathology characterized by severe bile duct damage on day 3 and biliary sclerosis on day 9. Consumption of I3C on its own did not affect the weight of the animals or liver function as reflected by biochemical indices or pathology.

In rats which received the combination of ET-743 and I3C (0.1%), plasma bilirubin levels were moderately reduced compared to rats which received ET-743 alone. On day 9 the difference was significant. I3C at this dose did not decrease AST and ALP activity, when compared with levels induced by ET-743 alone. Pathological manifestations of liver damage were only marginally less severe than in rats which received ET-743 only.

In contrast, a dietary concentration of 0.5% I3C protected rats from ET-743 induced changes as adjudged by plasma indicators of hepatic damage or hepatic pathology. Rats which had consumed I3C showed significantly less biliary damage compared with rats given ET-743 alone, and there was only slight irregular bile duct epithelium and very sparse degenerate biliary cells at 3 days and mild peribiliary fibrosis in rats on I3C at 9 days. Thus the protection afforded by 0.5% I3C was complete as reflected by the biochemical indices, and substantial, albeit not complete, when adjudged on the basis of liver pathology.

Concerning di-indolyl methane, DIM has been suggested to be responsible for, or contribute to, pharmacological effects of I3C after oral administration. Therefore we explored whether DIM might be responsible, at least in part, for the hepatoprotection afforded by I3C. To that end I3C was replaced with DIM (0.2% in the diet) and its ability to protect rat livers against the detrimental activity of ET-743 was studied. DIM pretreatment failed to ameliorate the biochemical changes elicited by ET-743. Likewise, on pathological investigation there was no difference between livers from animals which received ET-743 alone and those on DIM with ET-743.

Taken together, these results show that I3C, preferably at an oral dose of approximately 400 mg/kg, but not its metabonate DIM, protects rat livers efficiently against ET-743-induced changes. However a fifth of this dose is insufficient for protection.

### Example 2

### Study of Antitumor Activity

For a hepatoprotection strategy involving I3C to be clinically feasible, it needs to be demonstrated that I3C does not adversely affect the antitumor activity of ET-743.

13762 tumor fragments (100-200 mg weight) were implanted (sc) into the flank of female Fischer rats (100-120 g). Also in this rat strain ET-743 (40 μg/kg, Lv.) alone has been shown to cause changes in plasma levels of liver-specific indicators and liver histopathology identical to those described for Wistar rats in Example 1. Properties of the 13762 tumour have previously been described for example in Braunschweiger P. G., and Schiffer L. M. 1980, J. Natl. Cancer Inst., 64: 671-674: “Growth kinetics of mammary tumor 13762 in rats previously cured by chemotherapy”. Tumor weight (TW) was calculated on each day via tumor diameters, using a Vernier caliper and the formula: TW (in mg)=tumor volume (mm3)=d²×D/2, in which d and D represent the shortest and longest diameter, respectively. Rats (9-10 per group) received I3C in their diet (0.5%) from the day of tumor implantation to the end of the experiment. ET-743 (40 μg/kg, i.v.) was administered on day 6 post tumor implantation.

We found that 0.5% dietary I3C afforded the same degree of protection against the pathological manifestations of ET-743 in the liver of this rat strain as in Wistar rat. Both I3C and ET-743 retarded tumour growth on their own, with tumor weight inhibition (TWI) values of 35 and 62%, respectively, on day 20, the day the experiment was terminated. Most importantly, the activity of the combination was not inferior to that of ET-743 alone, exemplified by a TWI of 67% on day 20. Tumors were excised and weighed at termination of the experiment, and the results support the conclusion that ET-743 and I3C alone decreased terminal tumor weight by 54 and 43%, respectively, whereas the combination elicited a 71% weight reduction.

### Example 3

### Study of ET-743 Levels in Liver and Plasma

We tested the hypothesis that I3C pretreatment alters clearance of ET-743 from plasma and liver in vivo. Blood samples and liver tissue were collected from rats before and up to 24 h after administration of ET-743 (40 μg/kg iv.) with or without treatment with I3C (0.5% in the diet) given from 6 days prior to ET-743. Blood samples (by cardiac puncture) and liver tissue were collected before and at 0.5, 1, 3, 6, 12 and 24 h post treatment. Blood was placed in heparinized tubes, and plasma was obtained by centrifugation. An aliquot (0.3 ml) was mixed with 0.7 ml ammonium acetate buffer (0.2 M, pH 5.0). Liver tissue was homogenized (1:1) in water. Levels of ET-743 were measured by HPLC coupled to electrospray ionization tandem mass spectrometry in the plasma and liver samples.

I3C did not alter ET-743 disposition in the plasma or liver markedly, consistent with its un-impeded antitumor activity. The mean values for the area under the plasma or liver concentration versus time curves (AUC, between 0 and 24 h after dosing) of ET-743 after administration of ET-743 alone or in combination with I3C were 4.7 and 1.9 ngxh/ml, respectively, for the plasma and 280 and 563 ngxh/ml, respectively, for the liver. Thus while I3C treatment decreased levels of ET-743 in the plasma, it did not decrease ET-743 levels in the liver, instead I3C elevated hepatic ET-743 levels.

1. The use of a cruciferous indole compound in the preparation of a medicament for reducing undesirable toxic side effects inherent in the anti-tumor therapy with ET-743.

2. The use of ET-743 in the preparation of a medicament for an effective treatment of a tumour by combination therapy, employing in said combination therapy ET-743 with a cruciferous indole compound.

3. The use according to claim 1 or 2, wherein the cruciferous indole compound and ET-743 form part of the same medicament.

4. The use according to claim 1 or 2, wherein the cruciferous indole compound and ET-743 form part of separate medicaments.

5. The use according to any of the preceding claim, wherein the cruciferous indole compound is indole-3-carbinol.

6. The use according to any of the preceding claim, wherein the combination therapy further employs another hepatoprotector.

7. The use according to claim 6, wherein the other hepatoprotector is dexamethasone.

8. The use according to any of the preceding claim wherein the patient has a cancer selected from sarcoma, osteosarcoma, ovarian cancer, breast cancer, NSCL carcinoma, melanoma, head and neck cancer, colorectal cancer, mesothelioma, renal cancer, endometrial cancer and lung cancer.

9. A method for reducing undesirable toxic side effects inherent in the anti-tumor therapy with ET-743 which comprises administering a cruciferous indole compound to a patient.

10. A method for an effective treatment of a tumour by combination therapy, employing in said combination therapy ET-743 with a cruciferous indole compound.

11. A method according to claim 9 or 10, wherein the cruciferous indole compound is administered to a patient who is going to be treated with ET-743.

12. The method according to claim 9 or 10, wherein the cruciferous indole compound and ET-743 form part of the same medicament.

13. The method according to claim 9 or 10, wherein the cruciferous indole compound and ET-743 form part of separate medicaments.

14. The method according to any of claims 9 to 13, wherein the cruciferous indole compound is indole-3-carbinol.

15. The method according to any of claims 9 to 14, wherein the combination therapy further employs another hepatoprotector.

16. The method according to claim 15, wherein the other hepatoprotector is dexamethasone.

17. The method according to any claims 9 to 16, wherein the patient has a cancer selected from sarcoma, osteosarcoma, ovarian cancer, breast cancer, NSCL carcinoma, melanoma, head and neck cancer, colorectal cancer, mesothelioma, renal cancer, endometrial cancer and lung cancer.

18. A composition for the treatment of cancer, which comprises ET-743 and a cruciferous indole compound.

19. The composition according to claim 18, wherein the cruciferous indole compound is indole-3-carbinol.
